(19) United States
(12) Patent Application Publication
(10) Pub. No.: US 2004/0230272 A1
(43) Pub. Date: Nov. 18, 2004

(54) Subcutaneous lead with temporary pharmacological agents

(76) Inventors: Adam W. Cates, Minneapolis, MN (US); Darrell Orvin Wagner, Isanti, MN (US); Curtis Charles Lindstrom, Roseville, MN (US); Ron Heil, Roseville, MN (US)

Correspondence Address: Crawford Maunu PLLC, Suite 390, 1270 Northland Drive, St. Paul, MN, 55120, US

(21) Appl. No.: 10/703,409

(22) Filed: Nov. 7, 2003

Related U.S. Application Data
(60) Provisional application No. 60/462,272, filed on Apr. 11, 2003.

(51) Int. Cl. A61N 1/05

(52) U.S. Cl. 607/120

(57) ABSTRACT

An implantable subcutaneous device and method employ a lead and/or electrode for cardiac monitoring and intervention. The device includes an implantable lead having a lead body, a subcutaneous electrode supported by the lead body, and a pharmacological agent provided on the implantable lead body and/or an inactive can portion. The pharmacological agent provides a temporary therapeutic treatment to subcutaneous non-intrathoracic tissue. A method of implanting subcutaneous leads involves providing a lead including a lead body, a subcutaneous electrode, and a pharmacological agent, and delivering the pharmacological agent to subcutaneous non-intrathoracic tissue surrounding the lead.

### RELATED APPLICATIONS

This application claims the benefit of Provisional Patent Application Ser. No. 60/462,272, filed on Apr. 11, 2003, to which priority is claimed pursuant to 35 U.S.C. §119(e) and which is hereby incorporated herein by reference.

### FIELD OF THE INVENTION

The present invention relates generally to leads for subcutaneously implantable cardioverters/defibrillators and monitors, and, more particularly, to subcutaneously implantable leads provided with pharmacological agents.

### BACKGROUND OF THE INVENTION

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses that are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating arrhythmias.

Implantable cardioverter/defibrillators (ICDs) have been used as an effective treatment for patients with serious cardiac arrhythmias. For example, a typical ICD includes one or more endocardial leads to which at least one defibrillation electrode is connected. Such ICDs are capable of delivering high-energy shocks to the heart, interrupting the ventricular tachyarrythmia or ventricular fibrillation, and allowing the heart to resume normal sinus rhythm. ICDs may also include pacing functionality.

Although ICDs are very effective at preventing Sudden Cardiac Death (SCD), most people at risk of SCD are not provided with implantable defibrillators. The primary reasons for this unfortunate reality include the limited number of physicians qualified to perform transvenous lead/electrode implantation, a limited number of surgical facilities adequately equipped to accommodate such cardiac procedures, and a limited number of the at-risk patient population that can safely undergo the required endocardial or epicardial lead/electrode implant procedure. For this reason, subcutaneous ICDs are being developed to overcome these issues.

For reasons stated above, and for other reasons which will become apparent to those skilled in the art upon reading the present specification, there is a need for systems and methods that provide for sensing cardiac activity and delivering defibrillation and/or pacing therapies without the need for endocardial or epicardial leads/electrodes. There is a particular need for subcutaneous leads that improve patient comfort, reduce morbidity, and improve surgical outcomes. The present invention fulfills these and other needs, and addresses deficiencies in known systems and techniques.

### SUMMARY OF THE INVENTION

The present invention is directed to subcutaneous leads, lead systems, and methods of using subcutaneous leads that improve patient comfort, reduce morbidity, and improve surgical outcomes by incorporating pharmacological agents. The device has an implantable lead including a lead body, an electrode supported by the lead body and a pharmacological agent provided on the lead body and/or inactive portion of the can. The pharmacological agent provides a temporary therapeutic treatment to subcutaneous non-intrathoracic tissue.

The lead may further include collars, coatings, membranes, porous regions, or other means of providing pharmacological agents on the lead. The lead may further include a plurality of pharmacological agents with different activities, effectiveness times, or locations of activity, as well as tiered therapies for both tiered activities and tiered dosages.

A method of implanting subcutaneous leads is described including providing a lead including a lead body, a subcutaneous electrode, and a pharmacological agent and delivering the pharmacological agent to subcutaneous non-intrathoracic tissue surrounding the lead body. The method may also include combinations of a plurality of pharmacological agents having combinations of temporary effectiveness time-periods. The method may further include the use of a sheath to implant leads, wherein the sheath may have temporary pharmacological agents.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIGS. 1A and 1B are views of a transthoracic cardiac stimulation device as implanted in a patient;

FIG. 2A illustrates a lead in accordance with the present invention, inserted in a dissected subcutaneous path leading from the can;

FIG. 2B illustrates various components of a transthoracic cardiac sensing and/or stimulation device positioned in accordance with embodiments of the invention;

FIG. 3 is a plan view of a lead enclosed within a sheath prior to deployment of pharmacological agents in accordance with the present invention;

FIG. 4 is a magnified view of a lead incorporating a pharmacological agent in accordance with the present invention;

FIG. 5 is a magnified view of another embodiment of a lead incorporating a pharmacological agent in accordance with the present invention;

FIG. 6 is a magnified view of another embodiment of a lead incorporating a pharmacological agent in accordance with the present invention;

FIG. 7 is a magnified view of another embodiment of a lead incorporating a pharmacological agent in accordance with the present invention; and

FIG. 8 is a magnified view of another embodiment of a lead incorporating a pharmacological agent in accordance with the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

A device employing an implantable lead implemented in accordance with the present invention can incorporate one or more of the features, structures, methods, or combinations thereof described herein below. For example, a subcutaneous cardiac monitor or stimulator can be implemented to include one or more of the advantageous features and/or processes described below. It is intended that such a device or method need not include all of the features and functions described herein, but can be implemented to include selected features and functions that provide for unique structures and/or functionality.

In general terms, an implantable lead implemented in accordance with the present invention can be used with a subcutaneous cardiac monitoring and/or stimulation device. One such device is an implantable transthoracic cardiac sensing and/or stimulation (ITCS) device that can be implanted under the skin in the chest region of a patient. The ITCS device may, for example, be implanted subcutaneously such that all or selected elements of the device are positioned on the patient's front, back, side, or other body locations suitable for sensing cardiac activity and delivering cardiac stimulation therapy. It is understood that elements of the ITCS device may be located at several different body locations, such as in the chest, abdominal, or subclavian region with electrode elements respectively positioned at different regions near, around, in, or on the heart.

The primary housing (e.g., the active or non-active can) of the ITCS device, for example, can be configured for positioning outside of the rib cage at an intercostal or subcostal location, within the abdomen, or in the upper chest region (e.g., subclavian location, such as above the third rib). In one implementation, one or more electrodes can be located on the primary housing and/or at other locations about, but not in direct contact with the heart, great vessel or coronary vasculature.

In another implementation, one or more leads incorporating electrodes can be located in direct contact with the heart, great vessel or coronary vasculature, such as via one or more leads implanted by use of conventional transvenous delivery approaches. In another implementation, for example, one or more subcutaneous electrode subsystems or electrode arrays can be used to sense cardiac activity and deliver cardiac stimulation energy in an ITCS device configuration employing an active can or a configuration employing a non-active can. Electrodes can be situated at anterior and/or posterior locations relative to the heart.

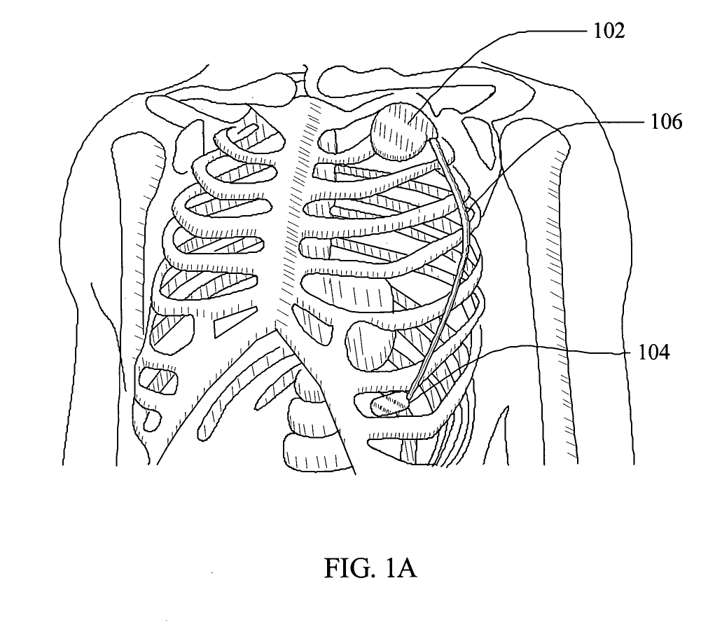

Referring now to FIGS. 1A and 1B of the drawings, there is shown a configuration of a transthoracic cardiac sensing and/or stimulation (ITCS) device having components implanted in the chest region of a patient at different locations by use of a dissection tool. In the particular configuration shown in FIGS. 1A and 1B, the ITCS device includes a housing 102 within which various cardiac sensing, detection, processing, and energy delivery circuitry can be housed. The housing 102 is typically configured to include one or more electrodes (e.g., can electrode and/or indifferent electrode). Although the housing 102 is typically configured as an active can, it is appreciated that a non-active can configuration may be implemented, in which case at least two electrodes spaced apart from the housing 102 are employed. An ITCS system according to this approach is distinct from conventional approaches in that it is preferably configured to include a combination of two or more electrode subsystems that are implanted subcutaneously.

In the configuration shown in FIGS. 1A and 1B, a subcutaneous electrode 104 can be positioned under the skin in the chest region and situated distal from the housing 102. The subcutaneous and, if applicable, housing electrode(s) can be positioned about the heart at various locations and orientations, such as at various anterior and/or posterior locations relative to the heart. The subcutaneous electrode 104 is electrically coupled to circuitry within the housing 102 via a lead assembly 106. One or more conductors (e.g., coils or cables) are provided within the lead assembly 106 and electrically couple the subcutaneous electrode 104 with circuitry in the housing 102. One or more sense, sense/pace or defibrillation electrodes can be situated on the elongated structure of the electrode support or the lead body 106, the housing 102, and/or the distal electrode assembly (shown as subcutaneous electrode 104 in the configuration shown in FIGS. 1A and 1B).

In one configuration, the lead assembly 106 is generally flexible. In another configuration, the lead assembly 106 is constructed to be somewhat flexible, yet has an elastic, spring, or mechanical memory that retains a desired configuration after being shaped or manipulated by a clinician. For example, the lead assembly 106 can incorporate a gooseneck or braid system that can be distorted under manual force to take on a desired shape. In this manner, the lead assembly 106 can be shape-fit to accommodate the unique anatomical configuration of a given patient, and generally retains a customized shape after implantation. Shaping of the lead assembly 106 according to this configuration can occur prior to, and during, ITCS device implantation.

In accordance with a further configuration, the lead assembly 106 includes a rigid electrode support assembly, such as a rigid elongated structure that positionally stabilizes the subcutaneous electrode 104 with respect to the housing 102. In this configuration, the rigidity of the elongated structure maintains a desired spacing between the subcutaneous electrode 104 and the housing 102, and a desired orientation of the subcutaneous electrode 104/housing 102 relative to the patient's heart. The elongated structure can be formed from a structural plastic, composite or metallic material, and comprises, or is covered by, a biocompatible material. Appropriate electrical isolation between the housing 102 and the subcutaneous electrode 104 is provided in cases where the elongated structure is formed from an electrically conductive material, such as metal.

In one configuration, the rigid electrode support assembly and the housing 102 define a unitary structure (i.e., a single housing/unit). The electronic components and electrode conductors/connectors are disposed within or on the unitary ITCS device housing/electrode support assembly. At least two electrodes are supported on the unitary structure near opposing ends of the housing/electrode support assembly. The unitary structure can have, for example, an arcuate or angled shape.

According to another configuration, the rigid electrode support assembly defines a physically separable unit relative to the housing 102. The rigid electrode support assembly includes mechanical and electrical couplings that facilitate mating engagement with corresponding mechanical and electrical couplings of the housing 102. For example, a header block arrangement can be configured to include both electrical and mechanical couplings that provide for mechanical and electrical connections between the rigid electrode support assembly and housing 102. The header block arrangement can be provided on the housing 102 or the rigid electrode support assembly, or both the housing 102 and the rigid electrode support assembly. Alternatively, a mechanical/electrical coupler can be used to establish mechanical and electrical connections between the rigid electrode support assembly and the housing 102. In such a configuration, a variety of different electrode support assemblies of varying shapes, sizes, and electrode configurations can be made available for physically and electrically connecting to a standard ITCS device.

It is noted that the electrodes and the lead assembly 106 can be configured to assume a variety of shapes. For example, the lead assembly 106 can have a wedge, chevron, flattened oval, or a ribbon shape, and the subcutaneous electrode 104 can comprise a number of spaced electrodes, such as an array or band of electrodes. Moreover, two or more subcutaneous electrodes 104 can be mounted to multiple electrode support assemblies 106 to achieve a desired spaced relationship amongst the subcutaneous electrodes 104. Accordingly, subcutaneous leads of the present invention can be shaped appropriately for specific electrodes or families of electrodes and electrode support assemblies.

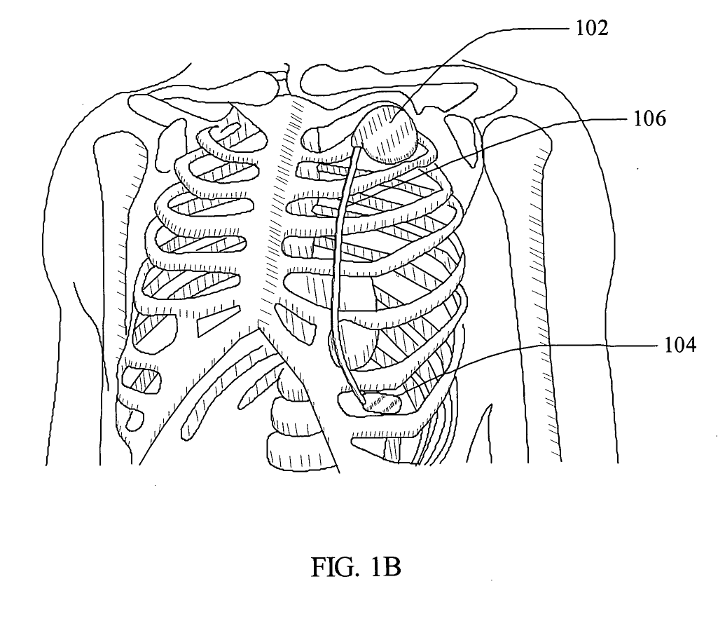

Referring now to FIG. 2A, an ITCS system 200 is illustrated including a can 250 with a lead 240 inserted into a dissection path 220. The lead 240 includes an electrode 230, here illustrated at the distal end of the lead 240. The dissection path 220 lies within the subcutaneous tissue of a patient as illustrated in FIGS. 1A and 1B.

Referring to FIG. 2B, a can electrode 502 is positioned on a housing 501 that encloses the ITCS device electronics. In one embodiment, the can electrode 502 comprises the entirety of the external surface of housing 501. In other embodiments, various portions of the housing 501 may be electrically isolated from the can electrode 502 or from tissue. For example, the active area of the can electrode 502 may comprise all or a portion of either the anterior or posterior surface of the housing 501 to direct current flow in a manner advantageous for cardiac sensing and/or stimulation.

The housing 501 may resemble that of a conventional implantable ICD, is approximately 20-100 cc in volume, with a thickness of 0.4 to 2 cm and with a surface area on each face of approximately 30 to 100 cm². As previously discussed, portions of the housing may be electrically isolated from tissue to optimally direct current flow. For example, portions of the housing 501 may be covered with a non-conductive, or otherwise electrically resistive, material to direct current flow. Suitable non-conductive material coatings include those formed from silicone rubber, polyurethane, or parylene, for example. These coating materials can be infused with one or more pharmacological agents as described below in greater detail.

In addition, or alternatively, all or portions of the housing 501 may be treated to change the electrical conductivity characteristics thereof for purposes of optimally directing current flow. Various known techniques can be employed to modify the surface conductivity characteristics of the housing 501, such as by increasing or decreasing surface conductivity, to optimize current flow. Such techniques can include those that mechanically or chemically alter the surface of the housing 501 to achieve desired electrical conductivity characteristics.

In the configuration shown in FIG. 2B, the ITCS device housing 501 containing the electronics (i.e., the can) is not used as an electrode. In this case, an electrode system comprising two electrode subsystems 508, 509 coupled to the housing 501 may be implanted subcutaneously in the chest region of the body, such as in the anterior thorax. The first and the second electrode subsystems 508, 509 are placed in opposition with respect to the ventricles of a heart 510, with the majority of the ventricular tissue of the heart 510 included within a volume defined between the electrode subsystems 508, 509. As illustrated in FIG. 2B, the first electrode system 508 is positioned superior to the heart 510 relative to a superior aspect of the heart 510, e.g., parallel to the left ventricular free wall. The second electrode system 509 is located inferior to the heart 510 and positioned in relation to an inferior aspect of the heart 510, e.g., parallel to the right ventricular free wall. A cable or wiring 506 conductively couples the electrode subsystems 508, 509 to the housing 501.

In this configuration, the first and the second electrode subsystems 508 and 509 may comprise any combination of electrodes used for sensing and/or electrical stimulation. In various configurations, the electrode subsystems 508, 509 may each be comprised of a single electrode or a combination of electrodes. The electrode or electrodes comprising the first and second electrode subsystems 508, 509 may include any combination of one or more coil electrodes, tip electrodes, ring electrodes, multi-element coils, spiral coils, spiral coils mounted on non-conductive backing, and screen patch electrodes, for example.

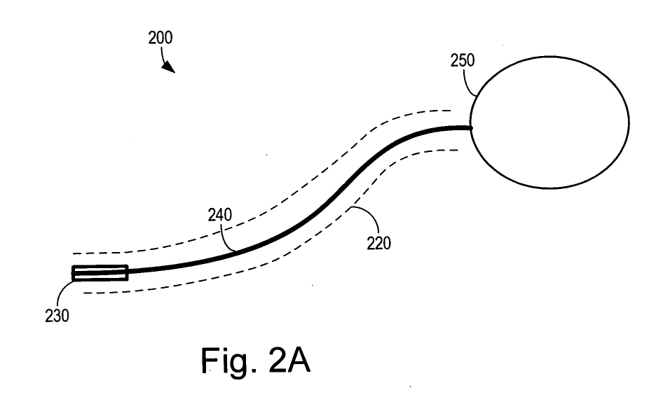

Referring to FIGS. 2A and 2B, the lead 240 and/or the electrode subsystems 508, 509 may be inserted into the dissection path 220 (FIG. 2A) by themselves, or may also be inserted with use of a sheath 320 as illustrated in FIG. 3. In FIG. 3, a proximal end of the lead 240 extends from the sheath 320, with the electrode 230 enclosed within the lumen of the sheath 320. The electrode 230 is illustrated with pharmacological agents 232 and 234 provided at distal and proximal ends of the electrode 230, respectively. It is understood that one, two, or more than two pharmacological agents can be provided at the electrode, and that provision of agents 232 and 234 is for illustrative purposes only.

The pharmacological agents 232 and 234 may be incorporated with the lead 240 via, for example, a collar, a porous region, a coating, or other suitable means. The lead 240 may be inserted into the dissection path 220 (FIG. 2A) inside the sheath 320. After properly locating the lead 240 within the subcutaneous tissue, the sheath 320 may be retracted from the subcutaneous tunnel thereby exposing the pharmacological agents 232 and 234 to the surrounding tissue and initiating a period of pharmacological activity by allowing drug(s) within pharmacological agents 232 and 234 to diffuse into the tissue surrounding the lead 240.

A non-limiting, non-exhaustive list of suitable pharmacological agents 232 and 234 includes analgesics, anesthetics, antibiotics, antiseptics, steroids, anti-inflammatory drugs, agents that promote hemostasis, agents that provide vasoconstriction, collagen, and agents that increase the rate of healing. Suitable analgesics or anesthetics may be, for example, aspirin, IBUPFOFEN, BUPIVACAINE, LIDOCAINE, MAPRIVACAINE and PROCAINE. Suitable steroids may be, for example, DEXAMETHASONE and BETAMETHASONE. A suitable pharmacological agent that provides vasoconstriction may be, for example, EPINEPHRINE. Suitable antibiotics or antiseptics may be, for example, VANCOMYCIN and CEFALOZIN. A suitable pharmacological agent that can increase the rate of healing may be, for example, stomach submucosa derived tissue such as disclosed in U.S. Pat. No. 6,099,567, which is hereby incorporated herein by reference.

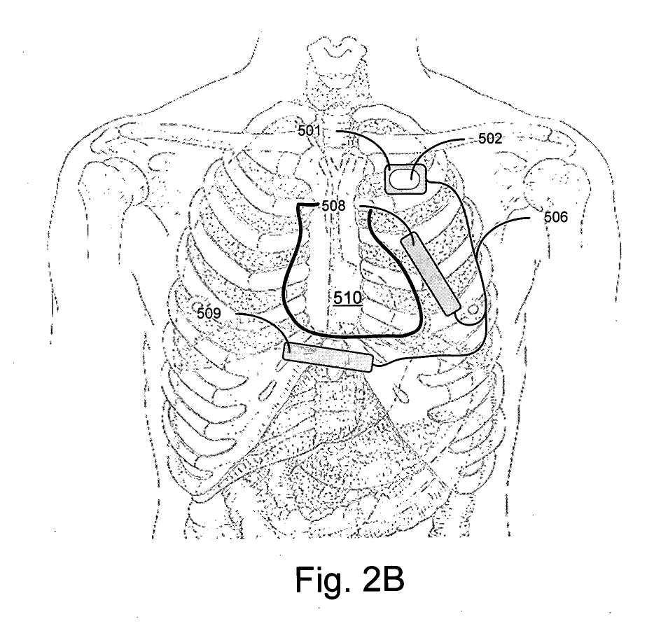

FIG. 4 illustrates an embodiment of the lead 240 with an electrode 230 and a coating 420. The coating 420 contains a pharmacological agent that is desired near the distal end of the lead 240. The coating 420 may be placed on the lead 240 by, for example, painting, spraying, dipping, vapor deposition, or other suitable means.

The lead 240 may be masked before applying the coating 420. For example, it may be desirable to place the coating 420 close to the electrode 230, as illustrated in FIG. 4, without covering the electrode 230. This may be accomplished by masking the electrode 230, spraying or dipping the coating 420, and then removing the masking, leaving the coating 420 in place without covering the electrode 230. It may be desirable to mask the electrode 230 if, for example, the pharmacological agent has an activity lasting more than one day. However, provision of the coating 420 covering the electrode 230 may alter the operating characteristics of the electrode 230.

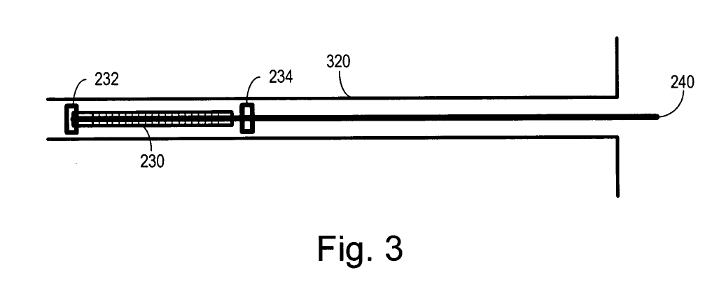

Referring now to FIG. 5, another embodiment of the lead 240 is illustrated, now with a second application 440 and a third application 460 of pharmacological agents in addition to the coating 420. It may be useful, for example, to periodically provide pharmacological agents along a portion of the length of the lead 240 to provide analgesia. Pharmacological agents may be applied at discrete locations, as illustrated in FIG. 5, or may be continuous along all or part of the lead 240. For example, an analgesic may be applied along a major portion (e.g. more than 25%) of lead 240, except on the electrode 230 where it may hinder electrical performance.

It may also be useful to apply a coating along a portion of the entire length of the lead 240 with, for example, an antiseptic, and also provide the coating 420 and/or second application 440 and/or third application 460 with, for example, an analgesic. This combination provides analgesia that diffuses locally to the dissected tissue surrounding the lead 240, while simultaneously providing an inhibition of infection along the dissection path. A temporary combination such as this may provide improved patient comfort and acceptance of the implant with improved outcomes and less morbidity from the procedure.

Pharmacological agents for use in accordance with the present invention provide a temporary effectiveness within the patient. The term temporary may have both quantitative and qualitative meanings. For example, direct analgesia may be desirable to tissue surrounding the dissection path immediately following the implantation procedure, to provide improved patient comfort between anesthesia during the procedure and any post-procedure medication. The quantitative time period of interest may be about one hour in this case. Qualitatively, for example, it would be beneficial to provide site-specific anesthesia or analgesia for the duration of time between when the general anesthesia wears off, and when a post-operative oral analgesic has reached an effective concentration level to provide pain relief, or to provide sufficient time duration such that oral analgesia is not necessary.

Another qualitative example of the term temporary as presently contemplated includes the use of an antiseptic agent delivered during the implantation procedure, and for a time period thereafter sufficient to reduce the morbidity of sepsis. The quantitative time frame for antisepsis may be on the order of hours, whereas for antibiotics, for example, a course of treatment often lasts ten days to several weeks. Qualitatively, it may be desirable to provide an antibiotic treatment from a portion of, or all of, the implanted device from the time of implantation until the probability of morbidity is diminished. Depending on patient variability and disease state, quantitatively, antibiosis may be desirable from about three days to more than about three weeks.

Other pharmacological agents in accordance with the present invention may have different quantitative and qualitative time frames associated with their effectiveness. For example, a xenograft collagen membrane that may be used to decrease the time necessary to heal from a tissue dissection, may be implanted as a permanent graft. However, the body will use the xenograft tissue as a scaffolding to increase the healing rate, but over time remodel the graft tissue to native tissue. The determination of when xenographic tissue is converted to native tissue is too imprecise to provide a precise quantitative time-frame applicable to all patients.

Therefore, for purposes herein, the term temporary is meant to encompass both the quantitative and qualitative aspects of pharmacological agents having an effectiveness for a limited time-period, the time period varying depending on the particular pharmacological activity desired. A non-exhaustive, non-limiting list of pharmacological activities includes: antisepsis, antibiosis, analgesia, anesthesia, vasoconstriction, and hemostasis.

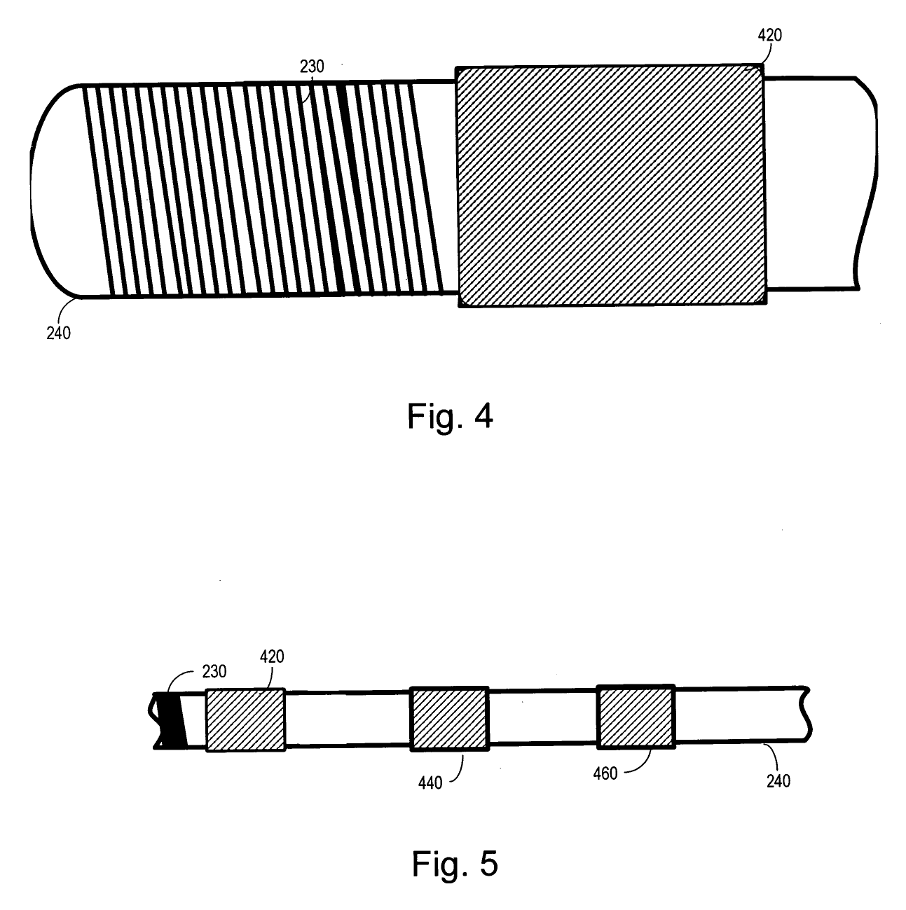

FIG. 6 illustrates another embodiment of the present invention. In FIG. 6, the lead 240 is shown to have a groove 610 providing a fixation point for a collar 620. Collar 620 may be, for example, a silicone collar impregnated with a pharmacological agent. Collars impregnated with pharmacological agents are known in the art such as, for example, collars described in U.S. Pat. No. 6,361,780 ('780) hereby incorporated herein by reference. Activity periods and dosage delivery of pharmacological agents can be tailored to the application by appropriate manufacture of the collar 620.

Although the width of groove 610 is illustrated in FIG. 6 to be significantly larger than the width of the collar 620, any desirable relationship may be provided. For example, the collar 620 may be provided with a plurality of layers incorporating a plurality of pharmacological agents, and/or a plurality of pharmacological activities. This would provide the potential for a tiered delivery of drug activity. For example, the outer layer may provide an acute high dosage for a short duration, while subsequent layers provide a longer term delivery.

In another embodiment of the present invention, an outermost layer of the collar 620 may provide analgesia while subsequent layers provide antibiosis and healing accelerators. By varying the width of the groove 610, and fitment of the collar 620, diffusion of the pharmacological agents from the layers of the collar 620 can be controlled to provide combinations of tiered therapy.

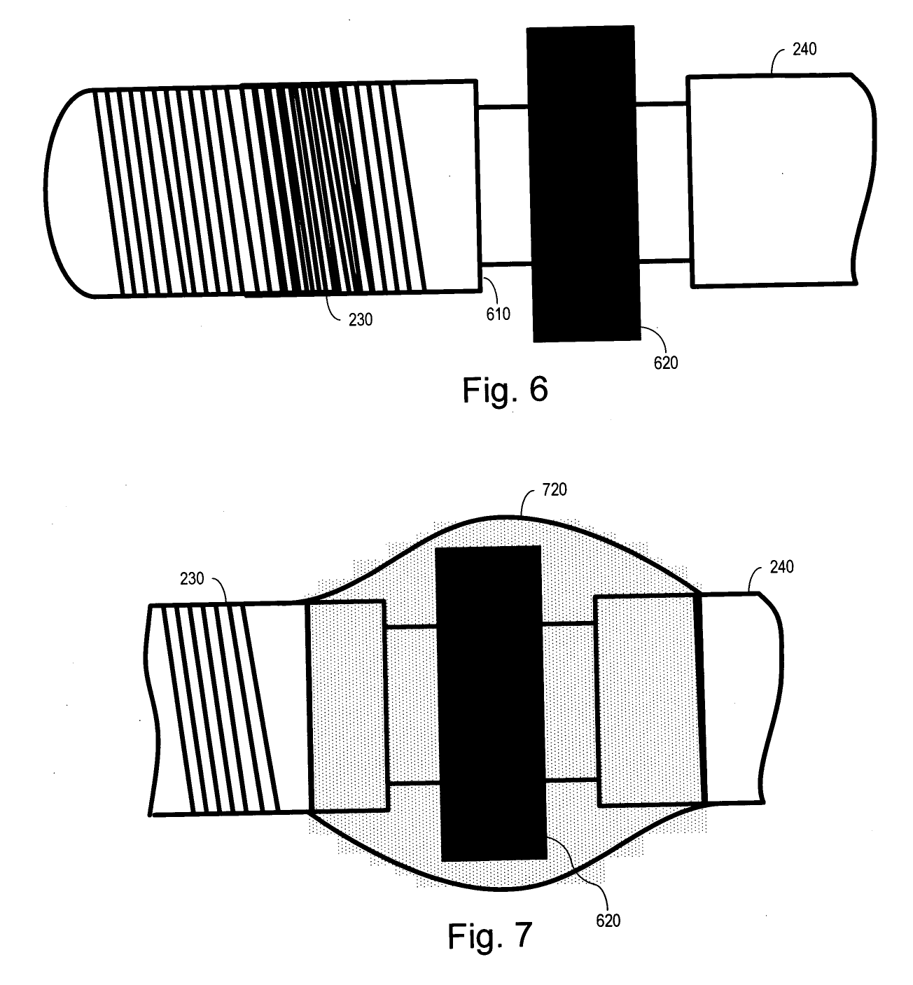

Yet another embodiment of the present invention is illustrated in FIG. 7. An encapsulation 720 encloses the collar 620 within the encapsulation 720. The encapsulation 720 may include a first pharmacological treatment wherein the encapsulation 720 dissolves away from the lead 240 over time. After a period of time, the encapsulation 720 is removed from the collar 620, exposing the collar 620 and providing a second pharmacological treatment. The first and second pharmacological treatments may vary by, for example, dosage intensities, pharmacological activities, therapy type, or other desired treatment. For example, the encapsulation 720 may be a non-dissolving semi-permeable membrane with the intent being the regulation of the diffusion rate from the collar 620 while protecting the collar 620 from the host body foreign object response.

In yet another embodiment, the lead 240 body material itself may be used to deliver a pharmacological agent. The normal lead 240 insulation may be used as the drug carrier. In practice, the polymeric insulation such as, for example, silicone can be swollen in an appropriate chemical agent such as, for example, alcohol, hexane, and/or Freon that also contains the pharmacological agent. By so treating the insulation, the pharmacological agent would diffuse into the insulation only to become trapped therein when the swelling agent is removed by, for example, evaporation, vacuum or other means known in the art. The treated insulation would then be used in the fabrication of the lead 240. Upon implantation of that lead 240, the superficial trapped pharmacological agent would first be released into the tissue, allowing deeper trapped pharmacological agents to migrate to the surface of the polymeric insulation for subsequent release into the tissue.

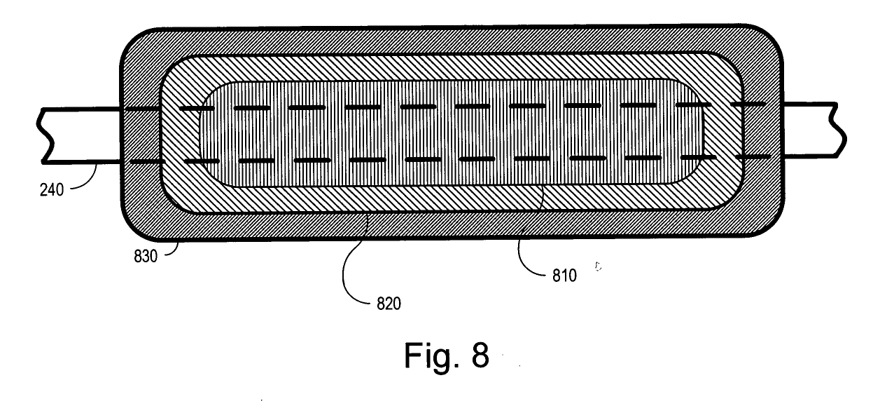

Referring now to FIG. 8, a three layer tiered pharmacological delivery approach is illustrated. The lead 240 is illustrated having a first layer 810, a second layer 820, and a third layer 830 on the lead 240. The layers 810, 820, and 830 are configured such that the pharmacological agent in the third layer 830 is delivered first as the third layer 830 is dissolved away and the pharmacological agent diffuses into the tissue and delivers its activity. After the third layer 830 is effectively removed, the second layer 820 is revealed. The pharmacological agent in the second layer 820 is delivered second as the second layer 820 is dissolved away and the pharmacological agent diffuses into the tissue and delivers its activity. After the second layer 820 is effectively removed, the first layer 810 is revealed. The pharmacological agent in the first layer 810 is delivered last as the first layer 810 is dissolved away and the pharmacological agent diffuses into the tissue and delivers its activity.

The layers 810, 820, and 830 may, for example, be continuous or discretely applied at one or a plurality of locations on the length of the lead 240, and have one or more drugs within each of the layers 810, 820 and 830. The layers 810, 820 and 830 may dissolve and/or may remain as a permanent scaffolding after releasing their pharmacological agents.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. An implantable lead, comprising:
a lead body;
an electrode coupled to the lead body, the electrode configured for subcutaneous non-intrathoracic placement within a patient; and
a pharmacological agent provided on the lead body, the pharmacological agent providing a temporary therapeutic treatment to subcutaneous non-intrathoracic tissue.

2. The lead according to claim 1, wherein the pharmacological agent's effectiveness is shorter than about one hour.

3. The lead according to claim 1, wherein the pharmacological agent's effectiveness is shorter than about one day.

4. The lead according to claim 1, wherein the pharmacological agent's effectiveness is shorter than about two weeks.

5. The lead according to claim 1, wherein the temporary therapeutic treatment is localized to an area substantially surrounding at least a portion of a subcutaneous dissection path.

6. The lead according to claim 1, wherein the pharmacological agent is provided at a plurality of locations on the lead body.

7. The lead according to claim 1, wherein the pharmacological agent comprises a membrane provided on the lead.

8. The lead according to claim 1, wherein the pharmacological agent is impregnated into a membrane provided on the lead.

9. The lead according to claim 1, wherein the lead comprises a collar, the pharmacological agent provided at the collar.

10. The lead according to claim 1, wherein the lead comprises a polymeric structure, the pharmacological agent infused within the polymeric structure.

11. The lead according to claim 1, wherein the lead comprises a porous region, the pharmacological agent at least partially filling pores of the porous region.

12. The lead according to claim 11, wherein the porous region comprises a doped polymer matrix.

13. The lead according to claim 1, wherein the pharmacological agent is disposed in a coating on the lead.

14. The lead according to claim 13, wherein the coating covers at least 25% of a surface area of the lead.

15. The lead according to claim 1, wherein the pharmacological agent comprises an analgesic or an anesthetic.

16. The lead according to claim 1, wherein the pharmacological agent comprises an antibiotic or an antiseptic.

17. The lead according to claim 1, wherein the pharmacological agent comprises a steroid or an anti-inflammatory agent.

18. The lead according to claim 1, wherein the pharmacological agent comprises an agent that promotes hemostasis or provides vasoconstriction.

19. The lead according to claim 1, wherein the pharmacological agent comprises collagen or an agent that increases a rate of healing.

20. An implantable system, comprising:
a can comprising an inactive area that is not electrically coupled to tissue substantially surrounding at least a portion of the inactive area;
a lead body;
an electrode coupled to the lead body, the electrode configured for subcutaneous non-intrathoracic placement within a patient; and
a pharmacological agent provided on at least a portion of the inactive area of the can, the pharmacological agent providing a temporary therapeutic treatment to subcutaneous non-intrathoracic tissue.

21. The system according to claim 20, wherein the pharmacological agent's effectiveness is shorter than about one hour.

22. The system according to claim 20, wherein the pharmacological agent's effectiveness is shorter than about one day.

23. The system according to claim 20, wherein the pharmacological agent's effectiveness is shorter than about two weeks.

24. The system according to claim 20, the can further comprising an insulating coating insulating a portion or all of the inactive area of the can from surrounding tissue, wherein the insulating coating comprises the pharmacological agent.

25. The system according to claim 20, wherein the pharmacological agent comprises a membrane provided on the can.

26. The system according to claim 20, wherein the pharmacological agent is impregnated into a membrane provided on the can.

27. The system according to claim 20, wherein the can comprises a porous region, the pharmacological agent at least partially filling pores of the porous region.

28. The system according to claim 27, wherein the porous region comprises a doped polymer matrix.

29. The system according to claim 20, wherein the pharmacological agent is disposed in a coating on the can.

30. The system according to claim 29, wherein the coating covers at least 25% of the inactive area of the can.

31. The system according to claim 20, wherein the pharmacological agent comprises an analgesic or an anesthetic.

32. The system according to claim 20, wherein the pharmacological agent comprises an antibiotic or an antiseptic.

33. The system according to claim 20, wherein the pharmacological agent comprises a steroid or an anti-inflammatory agent.

34. The system according to claim 20, wherein the pharmacological agent comprises an agent that promotes hemostasis or provides vasoconstriction.

35. The system according to claim 20, wherein the pharmacological agent comprises collagen or an agent that increases a rate of healing.

36. The system according to claim 20, comprising a plurality of pharmacological agents provided on at least a portion of the inactive area of the can, the pharmacological agents providing temporary therapeutic treatments to subcutaneous non-intrathoracic tissue.

37. An implantable lead, comprising:
a lead body;
an electrode coupled to the lead body, the electrode configured for subcutaneous non-intrathoracic placement within a patient; and
a plurality of pharmacological agents provided on the lead body, the pharmacological agents providing a plurality of temporary therapeutic treatments to subcutaneous non-intrathoracic tissue.

38. The lead according to claim 37, wherein the effectiveness of at least one of the pharmacological agents of the plurality of pharmacological agents is shorter than about one hour.

39. The lead according to claim 37, wherein the effectiveness of a first pharmacological agent of the plurality of pharmacological agents is shorter than about one hour and the effectiveness of a second pharmacological agent of the plurality of pharmacological agents is greater than about one hour and shorter than about two weeks.

40. The lead according to claim 37, wherein the effectiveness of at least two of the pharmacological agents of the plurality of pharmacological agents is shorter than about one hour.

41. The lead according to claim 37, wherein the effectiveness of at least two of the pharmacological agents of the plurality of pharmacological agents is greater than about one hour and shorter than about two weeks.

42. The lead according to claim 37, wherein the temporary therapeutic treatments are localized to an area substantially surrounding at least a portion of a subcutaneous dissection path.

43. The lead according to claim 37, wherein at least one of the pharmacological agents of the plurality of pharmacological agents is provided at a plurality of locations on the lead body.

44. The lead according to claim 37, wherein at least two pharmacological agents of the plurality of pharmacological agents are provided at a plurality of locations on the lead body.

45. The lead according to claim 37, wherein at least one of the pharmacological agents of the plurality of pharmacological agents is impregnated into a membrane provided on the lead.

46. The lead according to claim 37, wherein at least one of the pharmacological agents of the plurality of pharmacological agents comprises a membrane provided on the lead.

47. The lead according to claim 37, wherein the lead comprises a collar, wherein at least one of the pharmacological agents of the plurality of pharmacological agents is provided at the collar.

48. The lead according to claim 37, wherein the lead comprises a polymeric structure, at least one of the pharmacological agents of the plurality of pharmacological agents infused within the polymeric structure.

49. The lead according to claim 37, wherein the lead comprises a porous region, a first pharmacological agent of the plurality of pharmacological agents at least partially filling the pores of the porous region.

50. The lead according to claim 49, wherein the porous region comprises a doped polymer matrix.

51. The lead according to claim 50, wherein a second pharmacological agent of the plurality of pharmacological agents coats a portion of the lead.

52. The lead according to claim 51, wherein the second pharmacological agent covers at least a portion of the porous region, thereby delaying release of at least a portion of the first pharmacological agent until after a majority of an effectiveness period of the second pharmacological agent has elapsed.

53. The lead according to claim 51, wherein the second pharmacological agent comprises an analgesic or an anesthetic, and the first pharmacological agent comprises an antibiotic or an antiseptic.

54. The lead according to claim 51, wherein an effectiveness of the second pharmacological agent is shorter than about one day and an effectiveness of the first pharmacological agent is greater than about one day.

55. The lead according to claim 51, wherein the second pharmacological agent comprises an analgesic and an antiseptic, and the first pharmacological agent comprises an antibiotic.

56. The lead according to claim 37, wherein at least a first pharmacological agent of the plurality of pharmacological agents is provided in a coating on the lead.

57. The lead according to claim 56, wherein the coating covers at least 25% of a surface area of the lead.

58. The lead according to claim 56, wherein a second pharmacological agent of the plurality of pharmacological agents coats at least a portion of the first pharmacological coating.

59. The lead according to claim 58, wherein the second pharmacological agent comprises an analgesic or an anesthetic, and the first pharmacological agent comprises an antibiotic or an antiseptic.

60. The lead according to claim 58, wherein an effectiveness of the second pharmacological agent is shorter than about one day and an effectiveness of the first pharmacological agent is greater than about one day.

61. The lead according to claim 37, wherein at least one of the pharmacological agents of the plurality of pharmacological agents comprises an analgesic or an anesthetic.

62. The lead according to claim 37, wherein at least one of the pharmacological agents of the plurality of pharmacological agents comprises an antibiotic or an antiseptic.

63. The lead according to claim 37, wherein at least one of the pharmacological agents of the plurality of pharmacological agents comprises a steroid or an anti-inflammatory agent.

64. The lead according to claim 37, wherein at least one of the pharmacological agents of the plurality of pharmacological agents comprises an agent that promotes hemostasis or provides vasoconstriction.

65. The lead according to claim 37, wherein at least one of the pharmacological agents of the plurality of pharmacological agents comprises collagen or an agent that increases a rate of healing.

66. The lead according to claim 37, wherein a first pharmacological agent of the plurality of pharmacological agents provides a first dosage of the first pharmacological agent, and a second pharmacological agent of the plurality of pharmacological agents provides a second dosage of the first pharmacological agent.

67. An implantable lead system, comprising:
a lead comprising a lead body and an electrode, the lead configured for subcutaneous non-intrathoracic placement within a patient;
a delivery apparatus comprising a sheath configured to introduce the lead to a desired subcutaneous non-intrathoracic location within the patient; and
a pharmacological agent provided on the sheath.

68. The lead system according to claim 67, wherein the pharmacological agent's effectiveness is shorter than about one hour.

69. The lead system according to claim 67, wherein the pharmacological agent's effectiveness is shorter than about one day.

70. The lead system according to claim 67, wherein the pharmacological agent's effectiveness is shorter than about two weeks.

71. The lead system according to claim 67, wherein the pharmacological agent is provided at a plurality of locations on the sheath.

72. The lead system according to claim 67, wherein the sheath comprises a polymeric structure, the pharmacological agent infused within the polymeric structure.

73. The lead system according to claim 67, wherein the sheath comprises a porous region, the pharmacological agent at least partially filling the pores of the porous region.

74. The lead system according to claim 73, wherein the porous region comprises a doped polymer matrix.

75. The lead system according to claim 67, wherein the pharmacological agent is provided in a coating on the sheath.

76. The lead system according to claim 75, wherein the coating covers at least 25% of a surface area of the sheath.

77. The lead system according to claim 67, wherein the pharmacological agent comprises an analgesic or an anesthetic.

78. The lead system according to claim 67, wherein the pharmacological agent comprises an antibiotic or an antiseptic.

79. The lead system according to claim 67, wherein the pharmacological agent comprises a steroid or an anti-inflammatory agent.

80. The lead system according to claim 67, wherein the pharmacological agent comprises an agent that promotes hemostasis or provides vasoconstriction.

81. The lead system according to claim 67, further comprising a pharmacological agent provided on the lead body, the pharmacological agent providing a temporary therapeutic treatment to subcutaneous non-intrathoracic tissue.

82. A method of lead implantation, comprising:
delivering a lead into subcutaneous non-intrathoracic tissue, the lead comprising a lead body, an electrode, and a temporary pharmacological agent on the lead; and
delivering the pharmacological agent from the lead to subcutaneous non-intrathoracic tissue substantially surrounding at least a portion of the lead.

83. The method according to claim 82, wherein the pharmacological agent's effectiveness is shorter than about one hour.

84. The method according to claim 82, wherein the pharmacological agent's effectiveness is shorter than about one day.

85. The method according to claim 82, wherein the pharmacological agent's effectiveness is shorter than about two weeks.

86. The method according to claim 82, wherein the pharmacological agent is delivered through a diffusion process.

87. The method according to claim 82, wherein delivering the pharmacological agent comprises delivering a first agent having a first activity and delivering a second agent having a second activity.

88. The method according to claim 82, wherein delivering the pharmacological agent comprises delivering a first agent at a first dosage and delivering the first agent at a second dosage.

89. The method according to claim 88, wherein the first activity occurs for shorter than about one hour and the second activity occurs for longer than about one hour.

90. The method according to claim 88, wherein the first activity comprises analgesia or anesthesia and the second activity comprises antisepsis or antibiosis.

91. The method according to claim 88, wherein the first activity lasts shorter than about one day and the second activity lasts longer than about one day.

92. The method according to claim 82, further comprising:
providing a removable sheath having a lumen;
advancing the lead through the lumen; and
retracting the sheath away from the lead.

93. An implantable lead, comprising:
a lead body;
an electrode coupled to the lead body, the electrode configured for subcutaneous non-intrathoracic placement in a patient; and
means for delivering a pharmacological agent to subcutaneous non-intrathoracic tissue.

94. The lead according to claim 93, wherein the delivering means comprises a first means for delivering a first pharmacological agent having a first effectiveness time period and a second means for delivering a second pharmacological agent having a second effectiveness time period.

95. The lead according to claim 94, wherein the first pharmacological agent comprises an analgesic or an anesthetic, and the second pharmacological agent comprises an antibiotic or an antiseptic.

96. The lead according to claim 93, wherein the pharmacological agent comprises an analgesic or an anesthetic.

97. The lead according to claim 93, wherein the pharmacological agent comprises an antibiotic or an antiseptic.

98. The lead according to claim 93, wherein the pharmacological agent comprises a steroid or an anti-inflammatory agent.

99. The lead according to claim 93, wherein the pharmacological agent comprises an agent that promotes hemostasis or provides vasoconstriction.

100. The lead according to claim 93, wherein the pharmacological agent comprises collagen or an agent that increases a rate of healing.
